(19) United States
(12) Patent Application Publication
(10) Pub. No.: US 2006/0052373 A1
(43) Pub. Date: Mar. 9, 2006

(54) Combination of dopamine agonists and aralkyl and aralkylidene heterocyclic lactams and imides

(75) Inventors: Paul W. Glue, Flemington, NJ (US); Gerard J. Marek, Indianapolis, IN (US); Mario D. Saltarelli, Lake Bluff, IL (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/991,046

(22) Filed: Nov. 17, 2004

Related U.S. Application Data
(60) Provisional application No. 60/525,302, filed on Nov. 26, 2003.

(51) Int. Cl. A61K 31/541

(52) U.S. Cl. 514/227.5

(57) ABSTRACT

The present invention relates to a pharmaceutical composition containing (i) a dopamine agonist or a pharmaceutically acceptable salt thereof,
(ii) a compound of the formula I 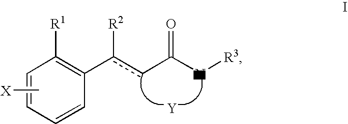 as defined in the specification, and optionally (iii) a pharmaceutically acceptable carrier, and to their medicinal use. These compositions are useful as psychotherapeutic agents.

### BACKGROUND OF THE INVENTION

The present invention relates to pharmaceutical compositions containing dopamine agonists or pharmaceutically acceptable salts thereof and aralkyl and aralkylidene heterocyclic lactams and imides or pharmaceutically acceptable salts thereof, and to their medicinal use. The aralkyl and aralkylidene heterocyclic lactams and imides include selective agonists and antagonists of serotonin 1 (5-HT₁) receptors, specifically, of one or both of the 5-HT_{1A} and 5-HT_{1B} receptors.

U.S. Pat. No. 6,380,186 and U.S. Patent Publication Nos. 2002/0091119, the contents of which are incorporated by reference herein, describe the aralkyl and aralkylidene heterocyclic lactams and imides which are used in the compositions of the present invention.

Dopamine agonists exhibit positive activity against disorders or conditions such as Parkinson's disease and depression, including major depression, as described in Depress. Anx., Vol. 11, pp. 58-65 (2000), and Pharmacopsychiatry, Vol. 34, pp. 137-141 (2001).

### SUMMARY OF THE INVENTION

The present invention relates to a pharmaceutical composition useful for example for treating a disorder or condition selected from the group consisting of hypertension, depression, generalized anxiety disorder, phobias, posttraumatic stress syndrome, avoidant personality disorder, sexual dysfunction, eating disorders, obesity, chemical dependencies, cluster headache, migraine, pain, Alzheimer's disease, obsessive-compulsive disorder, panic disorder, memory disorders, Parkinson's diseases, endocrine disorders, vasospasm, cerebellar ataxia, gastrointestinal tract disorders, negative symptoms of schizophrenia, premenstrual syndrome, Fibromyalgia Syndrome, stress incontinence, Tourette syndrome, trichotillomania, kleptomania, male impotence, cancer, chronic paroxysmal hemicrania and headache in a mammal, preferably a human, which pharmaceutical composition comprises
(i) a dopamine agonist or a pharmaceutically acceptable salt thereof,
(ii) a compound of the formula I 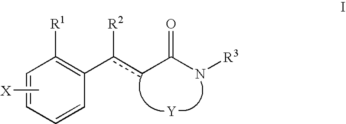
   wherein R¹ is a group of the formula G¹, G², G³, G⁴, G⁵, G⁶ or G⁷ depicted below, 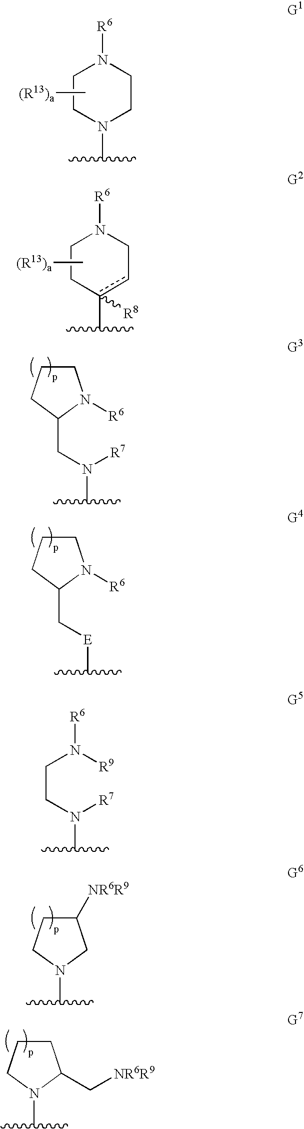
   a is zero to eight;
each R¹³ is, independently, (C₁-C₄)alkyl or a (C₁-C₄)methylene bridge from one of the ring carbons of the piperazine or piperidine ring of G¹ or G², respectively, to the same or another ring carbon or a ring nitrogen of the piperazine or piperidine ring of G¹ or G², respectively, having an available bonding site, or to a ring carbon of R⁶ having an available bonding site;
E is oxygen, sulfur, SO or SO₂;
X is hydrogen, chloro, fluoro, bromo, iodo, cyano, (C₁-C₆)alkyl, hydroxy trifluoromethyl, (C₁-C₆)alkoxy, —SOₜ (C₁-C₆)alkyl wherein t is zero, one or two, —CO₂R¹⁰ or —CONR¹¹R¹².
Y is an optionally substituted (C₁-C₄) heteroalkyl bridge that, together with the atoms to which it is attached, forms a five to seven membered heterocycle containing two to four heteroatoms selected from the group consisting of 1,3-oxazolidin-4-on-5-yl, 1,3-oxazolidin-2,4-dion-5-yl, 4,5-dihydro-1,2-oxazolidin-3-on-4-yl, 1,3-thiazolidin-4-on-5-yl, 1,3-thiazolidin-2,4-dion-5-yl, 1,3-pyrazolidin-4-on-5-yl, 1,3-imidazolidin-2,4-dion-5-yl, 1,2-pyrazolidin-3-on-4-yl, 1,2-thiazolidin-1,1,3-trion-4-yl, 1,2-thiazolidin-3-on-4-yl, tetrahydro-1,2-oxazin-3-on-4-yl, tetrahydro-1,3-oxazin-4-on-5-yl, tetrahydro-1,3-oxazin-2,4-dion-5-yl, morpholin-3-on-2-yl, morpholin-3,5-dion-2-yl, 2,3-dihydro-1,4-oxazin-3-on-2-yl, tetrahydro-1,3-thiazin-4-on-5-yl, tetrahydro-1,3-thiazin-2,4-d ion-5-yl, tetrahydro-1,2-thiazin-3-on-4-yl, thiomorpholin-3-on-2-yl, thiomorpholin-3,5-dion-2-yl, 2,3-dihydro-1,4-thiazin-3-on-2-yl, hexahydro-1,2-diazin-3-on-4-yl, 4,5-dihydro-2H-pyridazin-3-on-4-yl, hexahydro-1,3-diazin-4-on-5-yl, hexahydro-1,3-diazin-2,4-dion-5-yl, piperazin-2-on-3-yl, piperazin-2,6-dion-3-yl, tetrahydro-1,3,4-thiadiazin-5-on-6-yl, 5,6-dihydro-1,3,4-thiadiazin-5-on-6-yl, 1,3,4-oxadiazin-5-on-6-yl, 5,6-dihydro-1,2,4-oxadiazin-5-on-6-yl, tetrahydro-1,2,4-oxadiazin-5-on-6-yl, 1,2,4-triazin-5-on-6-yl, tetrahydro-1,2,4-oxadiazin-5-on-6-yl, 5,6-dihydro-1-2,4-oxadiazin-5-on-6-yl, 1,2,4-oxadiazin-3,5-dion-6-yl, 1,2,4-trazin-6-on-5-yl, hexahydro-1,2-oxazepin-3-on-2-yl, hexahydro-1,3-oxazepin-4-on-5-yl, hexahydro-1,4-oxazepin-3-on-2-yl, hexahydro-1,4-oxazepin-3,5-dion-2-yl, hexahydro-1,4-oxazepin-3,5-dion-6-yl, 2,3,5,6-tetrahydro-1-4-oxazepin-5,7-dion-6-yl, hexahydro-1,4-oxazepin-5-on-6-yl, hexahydro-1,3-oxazepin-2,4-dion-5-yl, hexahydro-1,2-thiazepin-3-on-4-yl, hexahydro-1,4-thiazepin-3-on-2-yl, 2,3,4,5-tetrahydro-1,4-thiazepin-3-on-2-yl, hexahydro-1,4-thiazepin-3,5-dion-2-yl, hexahydro-1,4-thiazepin-3,5-dion-6-yl, 2,3,6,7-tetrahydro-1,4-thiazepin-5-on-6-yl, 6,7-dihydro-1,4-thiazepin-5-on-6-yl, hexahydro-1,3-thiazepin-2,4-dion-5-yl, hexahydro-1,2-diazepin-3-on-4-yl, hexahydro-1,3-diazepin-2,4-dion-5-yl, hexahydro-1,4-diazepin-2-on-3-yl, hexahydro-1,4-diazepin-5-on-6-yl, hexahydro-1,4-diazepin-5,7-dion-6-yl, hexahydro-1,3,5-thiadiazepin-3-on-7-yl, 4,5,6,7-tetrahydro-1-3,5-thiadiazepin-6-on-7-yl, and 2,3,5,6-tetrahydro-1,2,4-triazepin-3,5-dion-7-yl; wherein the substituents on any of the carbon atoms capable of supporting an additional bond, of said (C₁-C₄) heteroalkyl bridge, are chloro, fluoro, (C₁-C₆)alkyl, (C₁-C₆)alkoxy, trifluoromethyl or cyano; wherein the substituents on any of the nitrogen atoms capable of supporting an additional bond, of said (C₁-C₄) heteroalkyl bridge, are (C₁-C₆)alkyl or trifluoromethyl,

R² is hydrogen, (C₁-C₄)alkyl, phenyl or naphthyl, wherein said phenyl or naphthyl may optionally be substituted with one or more substituents independently selected from the group consisting of chloro, fluoro, bromo, iodo, (C₁-C₆)alkyl, (C₁-C₆)alkoxy, trifluoromethyl, cyano and —SOₖ(C₁-C₆)alkyl wherein k is zero, one or two;
R³ is —(CH₂)ₘB, wherein m is zero, one, two or three and B is hydrogen, phenyl, naphthyl or a 5 or 6 membered heteroaryl group containing from one to four hetero-atoms in the ring, and wherein each of the foregoing phenyl, naphthyl and heteroaryl groups may optionally be substituted with one or more substituents independently selected from the group consisting of chloro, fluoro, bromo, iodo, (C₁-C₆)alkyl, (C₁-C₆)alkoxy, (C₁-C₆) alkoxy-(C₁-C₆)alkyl-, trifluoromethyl, trifluoromethoxy, cyano, hydroxy, —COOH and —SOₙ(C₁-C₆)alkyl wherein n is zero, one or two;
R⁶ is selected from the group consisting of hydrogen, (C₁-C₆)alkyl optionally substituted with (C₁-C₆)alkoxy or one to three fluorine atoms, or [(C₁-C₄)alkyl]aryl wherein the aryl moiety is phenyl, naphthyl, or heteroaryl-(CH₂)_{q}—, wherein the heteroaryl moiety is selected from the group consisting of pyridyl, pyrimidyl, benzoxazolyl, benzothiazolyl, benzisoxazolyl and benzisothiazolyl and q is zero, one, two, three or four, and wherein said aryl and heteroaryl moieties may optionally be substituted with one or more substituents independently selected from the group consisting of chloro, fluoro, bromo, iodo, (C₁-C₆)alkyl, (C₁-C₆)alkoxy, trifluoromethyl, cyano and —SO_{g}(C₁-C₆)alkyl, wherein g is zero, one or two;
R⁷ is selected from the group consisting of hydrogen, (C₁-C₆)alkyl, [(C₁-C₄)alkyl]aryl wherein the aryl moiety is phenyl, naphthyl, or heteroaryl-(CH₂)ᵣ—, wherein the heteroaryl moiety is selected from the group consisting of pyridyl, pyrimidyl, benzoxazolyl, benzothiazolyl, benzisoxazolyl and benzisothiazolyl and r is zero, one, two, three or four, and wherein said aryl and heteroaryl moieties may optionally be substituted with one or more substituents independently selected from the group consisting of chloro, fluoro, bromo, iodo, (C₁-C₆)alkyl, (C₁-C₆)alkoxy, trifluoromethyl, —C(═O)(C₁-C₆)alkyl, cyano and —SO_{g}(C₁-C₆)alkyl, wherein j is zero, one or two;
or R⁶ and R⁷ taken together form a 2 to 4 carbon chain;
R⁸ is hydrogen or (C₁-C₃)alkyl;
R⁹ is hydrogen or (C₁-C₆)alkyl;
or R⁶ and R⁹, together with the nitrogen atom to which they are attached, form a 5 to 7 membered heteroalkyl ring that may contain from zero to four heteroatoms selected from the group consisting of nitrogen, sulfur and oxygen;
and p is one, two, or three;
each of R¹⁰, R¹¹ and R¹² is selected, independently, from the radicals set forth in the definition of R²; or R¹¹ and R¹², together with the nitrogen to which they are attached, form a 5 to 7 membered heteroalkyl ring that may contain from zero to four heteroatoms selected from the group consisting of nitrogen, sulfur and oxygen, and
the broken lines indicate optional double bonds, with the proviso that when the broken line in G² is a double bond that R is absent;
or a pharmaceutically acceptable salt thereof;
and optionally
(iii) a pharmaceutically acceptable carrier.

The present invention also relates to:
a method for treating a disorder or condition described herein in a mammal, preferably a human, comprising administering to a mammal in need of such treatment components (i) and (ii);
a pharmaceutical composition for treating a disorder or condition that can be treated by enhancing serotonergic neurotransmission in a mammal, preferably a human, comprising components (i), (ii) and (iii);
a method for treating a disorder or condition that can be treated by enhancing serotonergic neurotransmission in a mammal, preferably a human, comprising administering to a mammal in need of such treatment components (i) and (ii); and a method for enhancing serotonergic neurotransmission in a mammal, preferably a human, comprising administering to a mammal in need of such treatment components (i) and (ii).

In one embodiment of the composition and method of the invention, the compound of formula I or a pharmaceutically acceptable salt thereof is present in a serotonin receptor antagonizing or agonizing effective amount. In another embodiment of the composition and method of the invention, the amounts of (i) the dopamine agonist or a pharmaceutically acceptable salt thereof and (ii) the compound of the formula I or a pharmaceutically acceptable salt thereof are such that the combination of (i) and (ii) is effective in treating a disorder or condition.

The compound of formula I may be a 5-HT_{1B} antagonist, a 5-HT_{1A} antagonist, or a 5-HT_{1A} antagonist and a 5-HT_{1B} antagonist. The composition of the invention may further comprise a 5-HT_{1A} antagonist. For example, in one embodiment of the invention, the composition and method of the invention are useful for treating a disorder or condition that can be treated by enhancing serotonergic neurotransmission in a mammal, the compound of formula I is a 5-HT_{1B} antagonist, and (i) the dopamine agonist or a pharmaceutically acceptable salt thereof and (ii) the compound of formula I or a pharmaceutically acceptable salt thereof are combined with a 5-HT_{1A} antagonist, or a pharmaceutically acceptable salt thereof, wherein (i), (ii), and the 5-HT_{1A} antagonist or pharmaceutically acceptable salt thereof are present in amounts such that the combination of (i), (ii), and the 5-HT_{1A} antagonist is effective in treating a disorder or condition listed herein.

### DETAILED DESCRIPTION OF THE INVENTION

“Enhancing serotonergic neurotransmission,” as used herein, refers to increasing or improving the neuronal process whereby serotonin is released by a pre-synaptic cell upon excitation and crosses the synapse to stimulate or inhibit the post-synaptic cell.

“Chemical dependency,” as used herein, means an abnormal craving or desire for, or an addiction to a drug. Such drugs are generally administered to the affected individual by any of a variety of means of administration, including oral, parenteral, nasal or by inhalation. Examples of chemical dependencies treatable by the methods of the present invention are dependencies on alcohol, nicotine, cocaine, amphetamine and other psychostimulants, morphine, heroin and other opioid agonists, phenobarbital and other barbiturates, and benzodiazepines such as diazepam and others. “Treating a chemical dependency,” as used herein, means reducing or alleviating such dependency.

A “unit dosage form” as used herein is any form that contains a unit dose of the dopamine agonist or a pharmaceutically acceptable salt thereof, of the compound of formula I or a pharmaceutically acceptable salt thereof, or of the dopamine agonist or pharmaceutically acceptable salt thereof and the compound of formula I or pharmaceutically acceptable salt thereof. A unit dosage form may be, for example, a tablet or a capsule. A unit dose may be an amount which may be predetermined, for example, by a physician.

A “monoamine reuptake inhibitor” as used herein is a reuptake inhibitor of the monoamine serotonin, norepinephrine, dopamine or a combination thereof.

Examples of the disorders or conditions which may be treated by the methods, compositions and kits of this invention are as follows:
depression, including depression in cancer patients, depression in Parkinson's patients, Postmyocardial Infarction depression, Subsyndromal Symptomatic depression, depression in infertile women, pediatric depression, major depression, single episode depression, recurrent depression, child abuse induced depression, and post partum depression, DSM-IV major depression, treatment-refractory major depression, bipolar depression BP I, bipolar depression BP II;
phobias, including agoraphobia, social phobia and simple phobias;
sexual dysfunction, including premature ejaculation;
eating disorders, including anorexia nervosa and bulimia nervosa;
chemical dependencies, including addictions to alcohol, cocaine, heroin, phenolbarbitol, nicotine and benzodiazepines;
memory disorders, including dementia, amnestic disorders, and age-related cognitive decline (ARCD);
Parkinson's diseases, including dementia in Parkinson's disease, neuroleptic-induced parkinsonism and tardive dyskinesias;
endocrine disorders, including hyperprolactinaemia;
vasospasm, including a vasospasm in the cerebral vasculature;
gastrointestinal tract disorders, including gastrointestinal tract disorders involving changes in motility and secretion;
cancer, including small cell lung carcinoma;
headache, including headache associated with vascular disorders.

As used herein, “mammal” means any member of the class Mammalia. As an example, the mammal in need of the treatment may be a human. As another example, the mammal in need of the treatment may be a mammal other than a human.

Dopamine agonists which may be used in the present invention may include selective D2/D3 agonists and nonselective dopamine agonists, which may include dopamine/β-adrenergic receptor agonists; dopamine/opiate receptor agonists; and dopamine agonists/α₂-adrenergic antagonists.

Exemplary dopamine agonists which may be used in accordance with this invention include those having structure II shown below. 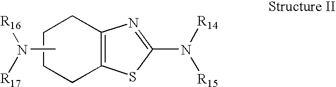
or a pharmaceutically acceptable acid addition salt thereof with an inorganic or organic acid, wherein R₁₄ represents a hydrogen atom, an alkyl group having 1 to 6 carbon atoms, an alkenyl or alkynyl group each having 3 to 6 carbon atoms, an alkanoyl group having 1 to 6 carbon atoms, a phenyl alkyl or phenyl alkanoyl group having 1 to 3 carbon atoms in the alkyl part, wherein each phenyl may be substituted by 1 or 2 halogen atoms, R₁₅ represents a hydrogen atom or an alkyl group with 1 to 4 carbon atoms, R₁₆ represents a hydrogen atom, an alkyl group with 1 to 7 carbon atoms, a cycloalkyl group having 3 to 7 carbon atoms, an alkenyl or alkynyl group having 3 to 6 carbon atoms, an alkanoyl group having 1 to 7 carbon atoms, a phenyl alkyl or phenyl alkanoyl group having 1 to 3 carbon atoms in the alkyl part, wherein each phenyl may be substituted by fluorine, chlorine or bromine atoms, R₁₇ represents a hydrogen atom, an alkyl group with 1 to 4 carbon atoms, an alkenyl or alkynyl group having 3 to 6 carbon atoms, or R₁₆ and R₁₇ together with the nitrogen atom between them represent a pyrrolidino, piperidino, hexamethyleneimino or morpholino group.

Compounds of structure II may be prepared, for example, as described in U.S. Pat. No. 4,886,812. An exemplary compound of structure II is pramipexole.

Exemplary dopamine agonists which may be used in accordance with this invention also include those having structure III shown below. 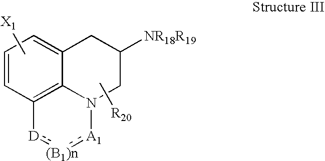
wherein R₁₈, R₁₉, and R₂₀ are each independently hydrogen, C₁₋₆ alkyl, C₂₋₆ alkenyl, or C₂₋₆ alkynyl, C₃₋₁₀ cycloalkyl, or R₁₈ and R¹ g are joined to form together with the nitrogen of NR₁₈R₁₉ a C₃₋₇ cyclic amine which can contain in addition to said nitrogen one or more heteroatoms selected from the group consisting of N, S and O; X₁ is hydrogen, C₁₋₆ alkyl, halogen, hydroxy, C₁₋₆ alkoxy, cyano, carboxamide, carboxyl, or C₁-C₆ alkoxycarbonyl; A₁ is SO₂, N, CH, CH₂, CHCH₃, C═O, C═S, CHSCH₃, C═NH, CNH₂, CNHCH₃, CNHCOOCH₃, or CNHCN; B₁ is CH₂, CH, C═O, N, NH or N—CH₃; n is 0 or 1; and D is CH, CH₂, C═O, O, N, NH or N—CH₃; or pharmaceutically acceptable salts thereof.

Compounds of structure III may be prepared, for example, as described in U.S. Pat. No. 5,273,975 incorporated by reference herein. An exemplary compound of structure III is sumanirole.

Examples of dopamine agonists which may be used in the present invention also include bromocriptine, lysuride, pergolide, aripiprazole and cabergoline.

The following are more specific embodiments of groups G¹ and G² of the compound of formula I

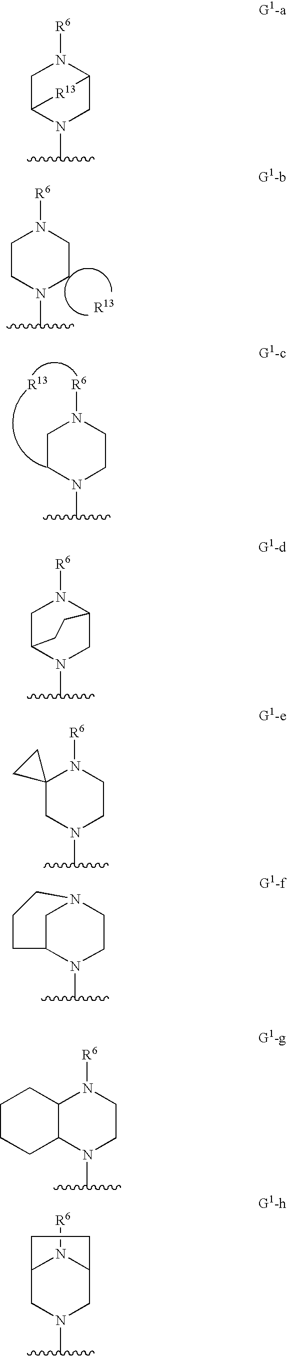

The compounds of formula I include all stereoisomers, such as cis and trans isomers, and all optical isomers of compounds of the formula I, such as R and S enantiomers, as well as racemic, diastereomeric and other mixtures of such isomers.

The compounds of formula I may contain C═C double bonds. When such bonds are present, the compounds of formula I exist as cis and trans configurations and as mixtures thereof.

Unless otherwise indicated, the alkyl and alkenyl groups referred to herein, as well as the alkyl moieties of other groups referred to herein, such as alkoxy, may be linear or branched, and they may also be cyclic, such as cyclopropyl, cyclobutyl, cyclopentyl, or cyclohexyl or be linear or branched and contain cyclic moieties. Unless otherwise indicated, halogen includes fluorine, chlorine, bromine, and iodine.

Preferred compounds of the formula I include those wherein R¹ is 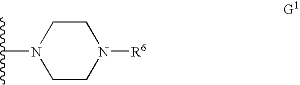
R⁶ is methyl and R² is hydrogen.

Preferred compounds of the formula I also include those wherein Y, together with the atoms to which it is attached, forms an optionally substituted five to seven membered heterocycle selected from the group consisting of 1,3 thiazolidin-2,4-dion-5-yl, 1,3 imidazolidin-2,4-dion-5-yl, thiomorpholin-3-on-2-yl or morpholin-3-on-2-yl.

Preferred compounds of the formula I also include those wherein R³ is optionally substituted phenyl or—(CH₂)-optionally substituted phenyl.

Examples of specific preferred compounds of the formula I are the following:
3-(4-chlorophenyl)-5-[2-(4-methylpiperazin-1-yl)-benzylidene]-imidazolidine-2,4-dione;
3-(4-chlorobenzyl)-5-[2-(4-methylpiperazin-1-yl)-benzylidene]-imidazolidine-2,4-dione;
3-(4-chlorobenzyl)-5-[2-(4-methylpiperazin-1-yl)-benzylidene]-thiazolidine-2,4-dione;
4-benzyl-2-[2-(4-methylpiperazin-1-yl)-benzylidene]-thiomorpholin-3-one;
4-(3,4-dichlorobenzyl)-2-[2-(4-methylpiperazin-1-yl)-benzylidene]-thiomorpholin-3-one;
3-(4-chlorophenyl)-5-[2-(4-methylpiperazin-1-yl)-benzylidene]-thiazolidine-2,4-dione;
3-(4-trifluoromethyphenyl)-5-[2-(4-methylpiperazin-1-yl)-benzylidene]-thiazol id ine-2,4-dione;
2-[2-(4-methylpiperazin-1-yl)-benzyl idene]-4-(4-trifluoromethylphenyl)-thiomorpholin-3-one;
2-[2-(4-methylpiperazin-1-yl)-benzylidene]-thiomorpholin-3-one;
4-(3,4-dichlorophenyl)-2-[2-fluoro-6-(4-methylpiperazin-1-yl)-benzylidene]-thiomorpholin-3-one;
4-(3,4-dichlorophenyl)-2-[2-(4-methylpiperazin-1-yl)-benzylidene]-morpholin-3-one;
4-(3,4-dichlorophenyl)-2-[2-(4-methylpiperazin-1-yl)-benzylidene]-thiomorpholin-3-one;
4-(3,4-dichlorophenyl)-2-[2-(4-methylpiperazin-1-yl)-benzyl]-thiomorpholin-3-one;
4-methyl-2-[2-(4-methylpiperazin-1-yl)-benzylidene]-thiomorpholin-3-one; and
4-(3,4-dichlorophenyl)-2-(2-piperazin-1-ylbenzylidene)-thiomorpholin-3-one;
4-Benzyl-2-[2-(4-methylpiperazin-1-yl)-benzylidene]-1,1-dioxothiomorpholin-3-one;
4-(3,4-Dichlorophenyl)-2-[3-fluoro-2-(4-methylpiperazin-1-yl)-benzylidene]-thiomorpholin-3-one;
4-(3,4-Dichlorophenyl)-2-[5-fluoro-2-(4-methylpiperazin-1-yl)-benzylidene]-thiomorpholin-3-one;
4-(3,4-Dichlorophenyl)-2-[2-(4-methylpiperazin-1-yl)-5-trifluoromethyl-benzylidene]-thiomorpholin-3-one;
4-(3,4-Dichlorophenyl)-2-{2-[4-(2-methoxyethyl)piperazin-1-yl]-benzyl idene} thiomorpholin-3-one;
4-(3,4-Dichlorophenyl)-2-[2-(4-isopropylpiperazin-1-yl)-benzylidene]-thiomorpholin-3-one;
4-(3,4-Dichlorophenyl)-2-[2-(4-ethylpiperazin-1-yl)-benzylidene]-thiomorpholin-3-one;
4-(4-Chlorophenyl)-2-[2-(4-methylpiperazin-1-yl)-benzylidene]-thiomorpholin-3-one;
4-(3-Chlorophenyl)-2-[2-(4-methylpiperazin-1-yl)-benzylidene]-thiomorpholin-3-one;
2-[2-Chloro-6-(4-methylpiperazin-1-yl)-benzylidene]-4-(3,4-dichlorophenyl)-thiomorpholin-3-one;
4-(3,4-Dichlorophenyl)-2-[2-(4-methylpiperazin-1-yl)-4-trifluoromethyl-benzyl idene]-thiomorpholin-3-one;
4-(3,4-Dichlorophenyl)-2-[2-(4-methylpiperazin-1-yl)-benzylidene]-1-oxo-thiomorpholin-3-one;
4-(3,4-Dichlorophenyl)-2-(5-fluoro-2-piperazin-1-yl-benzylidene)-thiomorpholin-3-one;
4-(3,4-Dichlorophenyl)-2-[3,6-difluoro-2-(4-methylpiperazin-1-yl)-benzylidene]-thiomorpholin-3-one;
4-(3,4-Dichlorophenyl)-2-[2-(3,5-dimethylpiperazin-1-yl)-benzylidene]-thiomorpholin-3-one;
4-Phenyl-2-[2-(3,4,5-trimethylpiperazin-1-yl)-benzylidene]-thiomorpholin-3-one;
2-[5-Fluoro-2-(4-methylpiperazin-1-yl)-benzylidene]-4-phenyl-thiomorpholin-3-one;
4-Benzo[1,3]dioxol-5-yl-2-[2-(3,5-dimethylpiperazin-1-yl)-benzyldene]-thiomorpholin-3-one;
2-[2-(4-tert-Butylpiperazin-1-yl)-benzyl idene]-4-(3,4-dichlorophenyl)-thiomorpholin-3-one;
3-(3,4-Dichlorophenyl)-5-[2-(4-methylpiperazin-1-yl)-benzylidene]-thiazolidin-4-one;
3-[4-(3,4-Dichlorophenyl)-3-oxo-thiomorpholin-2-ylidenemethyl]-6-dimethylamino-2-(4-methylpiperazin-1-yl)-benzonitrile;
5-[2-(4-Methylpiperazin-1-yl)-benzylidene]-2-phenylthiazolidin-4-one;
4-(3,4-Dichlorophenyl)-2-[2-(3,4,5-trimethylpiperazin-1-yl)-benzylidene]-thiomorpholin-3-one;
4-(3,4-Dichlorophenyl)-2-[5-methyl-2-(4-methylpiperazin-1-yl)-benzyl idene]-thiomorpholin-3-one;
2-[4-Chloro-2-(4-methylpiperazin-1-yl)-benzylidene]-4-(3,4-dichlorophenyl)-thiomorpholin-3-one;
4-(3,4-Difluorophenyl)-2-[2-(3,5-dimethylpiperazin-1-yl)-benzylidene]-thiomorpholin-3-one;
4-(2,4-Difluorophenyl)-2-[2-(3,5-dimethylpiperazin-1-yl)-benzylidene]-thiomorpholin-3-one;
2-[4-Bromo-2-(4-methylpiperazin-1-yl)-benzylidene]-4-(3,4-dichlorophenyl)-thiomorpholin-3-one;
4-(3,4-Dichlorophenyl)-2-[2-(1-methylpyrrolidin-2-ylmethoxy)-benzylidene]-thiomorpholin-3-one;
4-(3,5-Dichlorophenyl)-2-[2-(3,5-dimethylpiperazin-1-yl)-benzylidene]-thiomorpholin-3-one;
4-(3,4-Difluorophenyl)-2-[2-(3,4,5-trimethylpiperazin-1-yl)-benzylidene]-thiomorpholin-3-one;
4-(3,4-Dichlorophenyl)-2-[2-(octahydropyrido[1,2-a]pyrazin-2-yl)-benzylidene]-thiomorpholin-3-one,
2-[2-(4-Cyclopropylpiperazin-1-yl)-benzylidene]-4-pyridin-3-yl-thiomorpholin-3-one;
2-[2-(4-Cyclopropylpiperazin-1-yl)-benzylidene]-4-(3,4-difluorophenyl)-thiomorpholin-3-one;
2-[2-(4-Cyclopropylpiperazin-1-yl)-benzylidene]-4-(3,5-dichlorophenyl)-thiomorpholin-3-one;
4-(3,4-Difluorophenyl)-2-[2-(2,5-dimethylpiperazin-1-yl)-benzylidene]-thiomorpholin-3-one;
4-(3,5-Dichlorophenyl)-2-[2-(2,5-dimethylpiperazin-1-yl)-benzylidene]-thiomorpholin-3-one;
4-(3,4-Dichlorophenyl)-2-[2-(3-methylaminopyrrolidin-1-yl)-benzylidene]-thiomorpholin-3-one;
4-(3,4-Difluorophenyl)-2-[2-(2,4,5-trimethylpiperazin-1-yl)-benzylidene]-thiomorpholin-3-one;
4-Benzo[1,3]dioxol-5-yl-2-[2-(4-cyclopropylpiperazin-1-yl)-benzylidene]-thiomorpholin-3-one;
2-[2-(3,5-Dimethylpiperazin-1-yl)-benzylidene]-4-(4-fluorophenyl)-thiomorpholin-3-one:
4-Benzo[1,3]dioxol-5-yl-2-[2-(2,5-dimethylpiperazin-1-yl)-benzylidene]-thiomorpholin-3-one;
2-[2-(3,5-Dimethylpiperazin-1-yl)-benzylidene]-4-phenylthiomorpholin-3-one;
4-(3,4-Dichlorophenyl)-2-[2-(4-methylpiperazin-1-yl)-benzylidene]-thiomorpholin-3-one;
4-(3,4-Dichlorophenyl)-2-[2-(3-dimethylaminopyrrolidin-1-yl)-benzylidene]-thiomorpholin-3-one;
4-(3,4-Dichlorophenyl)-2-[2-(3-dimethylaminopyrrolidin-1-yl)-benzylidene]-thiomorpholin-3-one;
4-(3,4-Dichlorophenyl)-2-[2-(4-methyl-[1,4]diazepan-1-yl)-benzylidene]-thiomorpholin-3-one;
4-(3,4-Dichlorophenyl)-2-[2-(2,4,6-trimethylpiperazin-1-yl)-benzylidene]-thiomorpholin-3-one; and
2-[2-(4-Cyclopropylpiperazin-1-yl)-benzylidene]-4-(3,4-dichlorophenyl)-thiomorpholin-3-one;
   and the pharmaceutically acceptable salts of such compounds.

Other compounds of formula I include the following:
5-[2-(4-methylpiperazin-1-yl)-benzylidene]-thiazolidine-2,4-dione;
2-[2,4-dibromo-6-(4-methylpiperazin-1-yl)-benzylidene]-4-(3,4dichlorophenyl)-thiomorpholin-3-one;
4-(4-chlorophenyl)-2-[2-(4-methylpiperazin-1-yl)-benzylidene]-[1,4]oxazepan-3-one;
4-(4-chlorophenyl)-2-[2-(4-methylpiperazin-1-yl)-benzylidene]-[1,4,5]oxadiazepan-3-one;
4-(4-chlorophenyl)-2-[2-(4-methylpiperazin-1-yl)-benzyl idene]-[1,4]thiazepan-3-one;
4-(3,4-dichlorophenyl)-2-{2-[(2-dimethylaminoethyl)-methyl-amino]-benzylidene}-thiomorpholin-3-one;
4-(3,4-dichlorophenyl)-2-[2-(1-methylpiperidin-4-yl)-benzyl idene]-thiomorpholin-3-one;
4-(3,4-dichlorophenyl)-2-[2-(1,4-dimethylpiperidin-4-yl)-benzylidene]-thiomorpholin-3-one;
4-(3,4-dichlorophenyl)-2-[2-(4-methylpiperazin-1-yl)-benzylidene]-thiomorpholine-3,5-dione;
4-(3,4-dichlorophenyl)-2-[2-(2-dimethylaminoethoxy)-benzylidene]-thiomorpholin-3-one;
4-(3,4-dichlorophenyl)-2-[2-(4-isopropylpiperazin-1-yl)-benzylidene]-thiomorpholin-3-one;
4-(3,4-dichlorophenyl)-2-[2-(1-methylpyrrolidin-3-ylmethyl)-benzylidene]-thiomorpholin-3-one;
4-(3,4-dichlorophenyl)-2-{2-[methyl-(1-methylpyrrolidin-2-ylmethyl)-amino]-benzylidene}-thiomorpholin-3-one;
4-(3,4-dichlorophenyl)-2-[2-(1-methylpyrrolidin-2-ylmethoxy)-benzyl idene]-thiomorpholin-3-one;
4-(3,4-dichlorophenyl)-2-{2-[2-(1-methylpyrrolidin-2-yl)-ethyl]-benzylidene}-thiomorpholin-3-one;
1-(3,4-dichlorophenyl)-4-methyl-3-[2-(4-methylpiperazin-1-yl)-benzylidene]-piperazin-2-one;
4-methyl-3-[2-(4-methylpiperazin-1-yl)-benzylidene]-1-(4-trifluoromethylphenyl)-piperazin-2-one;
1-(4-chlorophenyl)-4-methyl-3-[2-(4-methylpiperazin-1-yl)-benzyl idene]-piperazin-2-one;
2-[2-(4-methylpiperazin-1-yl)-benzyl idene]-4-(4-trifluoromethylphenyl)-morpholin-3-one;
2-[4-fluoro-2-(4-methylpiperazin-1-yl)-benzylidene]-4-(4-trifluoromethylphenyl)-thiomorpholin-3-one;
2-[5-fluoro-2-(4-methylpiperazin-1-yl)-benzylidene]-4-(4-trifluoromethylphenyl)-thiomorpholin-3-one;
2-{1-[2-(4-methylpiperazin-1-yl)-phenyl]-ethylidene}-4-(4-trifluoromethylphenyl)-thiomorpholin-3-one;
2-[2-(4-methylpiperazin-1-yl)-benzyl]-4-(4-trifluoromethylphenyl)-thiomorpholin-3-one;
4-(4-chlorophenyl)-6-methyl-2-[2-(4-methylpiperazin-1-yl)-benzylidene]-thiomorpholin-3-one;
3-(4-chlorophenyl)-2,2-dimethyl-5-[2-(4-methylpiperazin-1-yl)-benzylidene]-thiazolidin-4-one;
4-(4-chlorophenyl)-2-[2-(4-methylpiperazin-1-yl)-benzylidene]-[1,4]oxazepan-3-one;
4-(4-chlorophenyl)-2-[2-(4-methylpiperazin-1-yl)-benzylidene]-4H-1,4]thiazin-3-one;
1-(4-chlorophenyl)-4,6,6-trimethyl-3-[2-(4-methylpiperazin-1-yl)-benzylidene]-piperazin-2-one;
1-(4-chlorophenyl)-4-methyl-3-[2-(4-methylpiperazin-1-yl)-benzylidene]-piperazin-2-one;
4-(4-chlorophenyl)-2-[2-(4-methylpiperazin-1-yl)-benzylidene]-morpholin-3-one;
3-(4-chlorophenyl)-5-[2-(4-methylpiperazin-1-yl)-benzylidene]-oxazolidin-4-one;
3-(4-chlorophenyl)-2,2-dimethyl-5-[2-(4-methylpiperazin-1-yl)-benzylidene]-imidazolidin-4-one; and
3-(4-chlorophenyl)-5-[2-(4-methylpiperazin-1-yl)-benzyl idene]-imidazolidin-4-one.

The compounds of the formula I which are basic in nature are capable of forming a wide variety of different salts with various inorganic and organic acids. Although such salts must be pharmaceutically acceptable for administration to animals, it is often desirable in practice to initially isolate a compound of the formula I from a reaction mixture as described in U.S. Pat. No. 6,380,186 as a pharmaceutically unacceptable salt and then simply convert the latter back to the free base compound by treatment with an alkaline reagent, and subsequently convert the free base to a pharmaceutically acceptable acid addition salt. The acid addition salts of the base compounds of formula I are readily prepared, for example, by treating the base compound with a substantially equivalent amount of the chosen mineral or organic acid in an aqueous solvent medium or in a suitable organic solvent such as methanol or ethanol. Upon careful evaporation of the solvent, the desired solid salt is obtained.

The acids which are used to prepare the pharmaceutically acceptable acid addition salts of the basic compounds of formula I and of the dopamine agonists are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [1,1′-methylene-bis-(2-hydroxy-3-naphthoate)]salts, as described, for example, in U.S. Pat. No. 6,380,186.

Those compounds of the formula I which are also acidic in nature, for example, where R³ includes a COOH or tetrazole moiety, are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of formula I are those which form non-toxic base salts with the herein described acidic compounds of formula I. These non-toxic base salts include those derived from such pharmacologically acceptable cations as sodium, potassium, calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum product yields.

The chemical bases that may be used as reagents to prepare pharmaceutically acceptable base salts of those compounds of formula I that are acidic in nature are those that form non-toxic base salts with such compounds. Such non-toxic base salts include, but are not limited to those derived from such pharmacologically acceptable cations such as alkali metal cations such as potassium and sodium and alkaline earth metal cations such as calcium and magnesium, ammonium or water-soluble amine addition salts such as N-methylglucamine (meglumine), and the lower alkanolammonium and other base salts of pharmaceutically acceptable organic amines, as described in U.S. Pat. No. 6,380,186.

Compounds of the formula I and their pharmaceutically acceptable salts are useful psychotherapeutics and are preferably potent agonists and/or antagonists of the serotonin 1A (5-HT_{1A}) and/or serotonin 1B (5-HT_{1B}) receptors. The compounds of the formula I are useful in the treatment of hypertension, depression, generalized anxiety disorder, phobias such as agoraphobia, social phobia and simple phobias, posttraumatic stress syndrome, avoidant personality disorder, sexual dysfunction such as premature ejaculation, eating disorders such as anorexia nervosa and bulimia nervosa, obesity, chemical dependencies such as addictions to alcohol, cocaine, heroin, phenolbarbitol, nicotine and benzodiazepines, cluster headache, migraine, pain, Alzheimer's disease, obsessive-compulsive disorder, panic disorder, memory disorders such as dementia, amnestic disorders, and age-related cognitive decline (ARCD), Parkinson's diseases such as dementia in Parkinson's disease, neuroleptic-induced parkinsonism and tardive dyskinesias, endocrine disorders such as hyperprolactinaemia, vasospasm, particularly in the cerebral vasculature, cerebellar ataxia, gastrointestinal tract disorders, such as involving changes in motility and secretion, negative symptoms of schizophrenia, premenstrual syndrome, Fibromyalgia Syndrome, stress incontinence, Tourette syndrome, trichotillomania, kleptomania, male impotence, cancer such as small cell lung carcinoma, chronic paroxysmal hemicrania and headache, such as headache associated with vascular disorders.

The compounds of formula I and the dopamine agonist of the composition of the invention may be further combined with one or more other therapeutic agents, for instance, different antidepressant agents such as tricyclic antidepressants such as amitriptyline, dothiepin, doxepin, trimipramine, butripyline, clomipramine, desipramine, imipramine, iprindole, lofepramine, nortriptyline or protriptyline, monoamine oxidase inhibitors such as isocarboxazid, phenelzine or tranylcyclopramine or monoamine reuptake inhibitors such as fluvoxamine, sertraline, fluoxetine or paroxetine, and/or with antiparkinsonian agents such as dopaminergic antiparkinsonian agents such as levodopa, preferably in combination with a peripheral decarboxylase inhibitor such as benserazide or carbidopa, and/or therapeutic agents which do not appreciably block monoamine uptake or affect monoamine oxidase such as mirtazapine, mianserin, bupropion, lithium salts, antiepileptic drugs such as caramazepine, valproate, lamotrigine, topiramate, gabapentin, pregabalin. It will be understood that the present invention covers the combination of a compound of general formula (I) or a pharmaceutically acceptable salt thereof with a dopamine agonist and with one or more such therapeutic agents, such as, for example, monoamine reuptake inhibitors, which include serotonin (5-HT) reuptake inhibitors. The monoamine reuptake inhibitor may have additional pharmacological properties, for example, antagonism of 5-HT_{1A} or 5-HT_{2A/C} receptors. Monoamine reuptake inhibition is readily determined by those skilled in the art according to standard assays such as those disclosed in U.S. Pat. No. 4,536,518. Sertraline, (1S-cis)-4-(3,4-dichlorophenyl)-1,2,3,4-tetrahydro-N-methyl-1-naphthalen-amine, which has the chemical formula C₁₇H₁₇Cl₂, is an exemplary monoamine reuptake inhibitor. Its synthesis is described in U.S. Pat. No. 4,536,518 assigned to Pfizer Inc.

The combination of the compounds of the formula I or the pharmaceutically acceptable salts thereof and a dopamine agonist or a pharmaceutically acceptable salt thereof is also referred herein to as “the active combination.”

The composition of the present invention may be formulated in a conventional manner using one or more pharmaceutically acceptable carriers. Thus, the active combinations of the invention may be formulated for oral, buccal, intranasal, parenteral (e.g., intravenous, intramuscular or subcutaneous) or rectal administration or in a form suitable for administration by inhalation or insufflation.

For oral administration, the pharmaceutical composition may take the form of, for example, tablets or capsules prepared by conventional means with pharmaceutically acceptable excipients such as binding agents such as pregelatinized maize starch, polyvinylpyrrolidone or hydroxypropyl methylcellulose; fillers such as lactose, microcrystalline cellulose or calcium phosphate; lubricants such as magnesium stearate, talc or silica; disintegrants such as potato starch or sodium starch glycolate; or wetting agents such as sodium lauryl sulphate. The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents such as sorbitol syrup, methyl cellulose or hydrogenated edible fats; emulsifying agents such as lecithin or acacia, non-aqueous vehicles such as almond oil, oily esters or ethyl alcohol; and preservatives such as methyl or propyl p-hydroxybenzoates or sorbic acid.

For buccal administration, the composition may take the form of tablets or lozenges formulated in conventional manner.

The composition of the present invention may be formulated for parenteral administration by injection, including using conventional catheterization techniques or infusion. Formulations for injection may be presented in unit dosage form, for example, in ampoules or in multi-dose containers, with an added preservative. The compositions of the present invention may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, for example, sterile pyrogen-free water, before use.

The composition of the present invention may also be formulated in rectal compositions such as suppositories or retention enemas, for example, containing conventional suppository bases such as cocoa butter or other glycerides.

For intranasal administration or administration by inhalation, the compositions of the present invention are conveniently delivered in the form of a solution or suspension from a pump spray container that is squeezed or pumped by the patient or as an aerosol spray presentation from a pressurized container or a nebulizer, with the use of a suitable propellant, for example, dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the compositions of the present invention. Capsules and cartridges, made, for example, from gelatin, for use in an inhaler or insufflator may be formulated containing a powder mix of an active compound and a suitable powder base such as lactose or starch.

An exemplary dose of the composition of the present invention for oral, parenteral or buccal administration to the average adult human for the treatment of the conditions referred to above, such as depression, is about 0.1 to about 200 mg of the active compound of formula I and of about 0.1 to about 300 mg of the dopamine agonist per unit dose which could be administered, for example, 1 to 4 times per day.

The composition of this invention may contain, for example, cabergoline, sumanirole or pramipexole or a pharmaceutically acceptable salt thereof as the dopamine agonist and 4-benzyl-2-[2-(4-methylpiperazin-1-yl)-benzylidene]-thiomorpholin-3-one, 4-(3,4-dichlorobenzyl)-2-[2-(4-methylpiperazin-1-yl)-benzylidene]-thiomorpholin-3-one, or 2-[2-(4-methylpiperazin-1-yl)-benzylidene]-4-(4-trifluoromethyl-phenyl)-thiomorpholin-3-one as the compound of formula I. An exemplary daily dose of the dopamine agonist in a pharmaceutical composition of this invention for oral, parenteral, rectal or buccal administration to the average adult human for the treatment of the conditions referred to above is from about 0.1 to about 300 mg of dopamine agonist per unit dose administered 1 to 3 times per day, such as about 0.1 mg to about 2 mg of pramipexole, preferably from about 0.25 mg to about 1.5 mg of pramipexole per unit dose which could be administered, for example 1 to 3 times per day. Another exemplary daily dose of the dopamine agonist is from about 1 mg to about 50 mg of sumanirole per unit dose which could be administered, for example 1 to 3 times per day. Another exemplary daily dose of the dopamine agonist is from about 1 mg to about 10 mg of cabergoline per unit dose which could be administered, for example 1 to 3 times per day. Exemplary and preferred doses for other dopamine agonists are determined on a compound by compound basis. 4-benzyl-2-[2-(4-methylpiperazin-1-yl)-benzylidene]-thiomorpholin-3-one, 4-(3,4-dichlorobenzyl)-2-[2-(4-methylpiperazin-1-yl)-benzylidene]-thiomorpholin-3-one, or 2-[2-(4-methylpiperazin-1-yl)-benzylidene]-4-(4-trifluoromethyl-phenyl)-thiomorpholin-3-one may each be present in an amount between about 0.1 to about 200 mg, preferably about 0.5 to about 10 mg.

In an exemplary embodiment, the composition of this invention contains about 0.5 mg of 4-benzyl-2-[2-(4-methylpiperazin-1-yl)-benzyl idene]-thiomorpholin-3-one as the compound of formula I and about 0.5 mg of pramipexole as the dopamine agonist and the composition is administered three times per day. In another exemplary embodiment, the composition of this invention contains about 0.5 mg of 4-(3,4-dichlorobenzyl)-2-[2-(4-methylpiperazin-1-yl)-benzylidene]-thiomorpholin-3-one as the compound of formula I and about 1.0 mg of pramipexole as the dopamine agonist and the composition is administered three times per day. In another exemplary embodiment, the composition of this invention contains about 0.5 mg of 2-[2-(4-methylpiperazin-1-yl)-benzylidene]-4-(4-trifluoromethylphenyl)-thiomorpholin-3-one as the compound of formula I and about 0.25 mg of pramipexole as the dopamine agonist and the composition is administered three times per day. In another exemplary embodiment, the composition of this invention contains about 0.5 mg of 4-benzyl-2-[2-(4-methylpiperazin-1-yl)-benzylidene]-thiomorpholin-3-one as the compound of formula I and about 10 mg of sumanirole as the dopamine agonist and the composition is administered three times per day. In another exemplary embodiment, the composition of this invention contains about 0.5 mg of 4-(3,4-dichlorobenzyl)-2-[2-(4-methylpiperazin-1-yl)-benzylidene]-thiomorpholin-3-one as the compound of formula I and about 20 mg of sumanirole as the dopamine agonist and the composition is administered three times per day. In another exemplary embodiment, the composition of this invention contains about 5 mg of 2-[2-(4-methylpiperazin-1-yl)-benzylidene]-4-(4-trifluoromethylphenyl)-thiomorpholin-3-one as the compound of formula I and about 0.5 mg of sumanirole as the dopamine agonist and the composition is administered three times per day. In another exemplary embodiment, the composition of this invention contains about 0.5 mg of 4-benzyl-2-[2-(4-methylpiperazin-1-yl)-benzylidene]-thiomorpholin-3-one as the compound of formula I and about 5 mg of cabergoline as the dopamine agonist and the composition is administered three times per day. In another exemplary embodiment, the composition of this invention contains about 0.5 mg of 4-(3,4-dichlorobenzyl)-2-[2-(4-methylpiperazin-1-yl)-benzylidene]-thiomorpholin-3-one as the compound of formula I and about 10 mg of cabergoline as the dopamine agonist and the composition is administered three times per day. In another exemplary embodiment, the composition of this invention contains about 5 mg of 2-[2-(4-methylpiperazin-1-yl)-benzylidene]-4-(4-trifluoromethylphenyl)-thiomorpholin-3-one as the compound of formula I and about 2.5 mg of cabergoline as the dopamine agonist and the composition is administered three times per day.

The dopamine agonist and the compound of formula I may be administered either alone or together with pharmaceutically acceptable carriers by either of the routes previously indicated, and such administration can be carried out in both single and multiple dosages. More particularly, the active combination can be administered in a wide variety of different dosage forms, i.e., they may be combined with various pharmaceutically-acceptable inert carriers in the form of tablets, capsules, lozenges, troches, hard candies, powders, sprays, aqueous suspension, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes. In general, the compounds of formula I are present in such dosage forms at concentration levels ranging from about 0.5% to about 90% by weight of the total composition, i.e., in amounts which are sufficient to provide the desired unit dosage, and a dopamine agonist is present in such dosage forms at concentration levels ranging from about 0.5% to about 90% by weight of the total composition, i.e., in amounts which are sufficient to provide the desired unit dosage.

The dopamine agonists and the compounds of formula I are preferably administered together. The dopamine agonists and the compounds of formula I may also be administered separately in either order, provided that after administration of the first of the two active ingredients, the second active ingredient is administered within 12 hours or less. When administered separately, the dopamine agonists and the compounds of formula I may be separately formulated and separately delivered in the same ways as described herein for the formulation of the compositions of the invention.

A preferred dose ratio of a dopamine agonist to an active compound of formula I in the composition of the present invention formulated for oral, parenteral or buccal administration to the average adult human for the treatment of the conditions referred to above is from about 0.001 to about 1000, preferably from about 0.001 to about 100.

Aerosol formulations for treatment of the conditions referred to above, for example, migraine, in the average adult human are preferably arranged so that each metered dose or “puff” of aerosol contains about 20 μg to about 1000 μg of the compound of formula I. The overall daily dose with an aerosol will be within the range about 100 μg to about 10 mg. Administration may be several times daily, for example 2, 3, 4 or 8 times, giving for example, 1, 2 or 3 doses each time. Aerosol formulations containing a compound of formula I and a dopamine agonist for treatment of the conditions referred to above in the average adult human are preferably arranged so that each metered dose or “puff” of aerosol contains about 100 μg to about 10,000 μg of the compound of formula I and about 100 μg to about 30,000 μg of the dopamine agonist. The overall daily dose with an aerosol will be within the range about 100 μg to about 20,000 mg of the compound of formula I and about 100 μg to about 60,000 mg of the dopamine agonist. Administration may be several times daily, for example 1, 3, 4 or 8 times, giving for example, 1, 2 or 3 doses each time.

The affinities of the compounds of the formula I for the various serotonin-1 receptors can be determined using standard radioligand binding assays as described in the literature. The 5-HT_{1A} affinity can be measured using the procedure of Hoyer et al. (Brain Res., 376, 85 (1986)). The 5-HT_{1B} affinity can be measured using the procedure of Heuring and Peroutka (J. Neurosci., 7, 894 (1987)). The activity of the compounds of the formula I at the 5-HT_{1B} binding site, the activity for 5-HT_{1A} binding ability, and the agonist and antagonist activities of the compounds of the formula I at 5-HT_{1A} and 5-HT_{1B} receptors may be determined as described in U.S. Pat. No. 6,380,186. All compounds of the formula I that were tested exhibited IC₅₀'s less than 0.60 μM for 5-HT_{1B} affinity and IC₅₀'s less than 1.0 μM for 5-HT_{1A} affinity. Similarly, The activity at the 5-HT_{1B} binding site, the activity for 5-HT_{1A} binding ability, and the agonist and antagonist activities of the compositions of the present invention may be determined using the procedures described for the compounds in formula I in U.S. Pat. No. 6,380,186.

The compositions of the invention can be evaluated as anti-migraine agents by testing the extent to which they mimic sumatriptan in contracting the dog isolated saphenous vein strip as described in P. P. A. Humphrey et al., Br. J. Pharmacol., 94, 1128 (1988). This effect can be blocked by methiothepin, a known serotonin antagonist. Sumatriptan is known to be useful in the treatment of migraine and produces a selective increase in carotid vascular resistance in the anesthetized dog. The pharmacological basis of sumatriptan efficacy has been discussed in W. Fenwick et al., Br. J. Pharmacol., 96, 83 (1989).

Activity of the active combinations as antidepressants and related pharmacological properties can be determined by methods (1)-(3) below, which are described in Koe, B. et al. Journal of Pharmacology and Experimental Therapeutics, 226 (3), 686-700 (1983). Specifically, activity can be determined by studying (1) their ability to affect the efforts of mice to escape from a swim-tank (Porsolt mouse “behavior despair” test), (2) their ability to potentiate 5-hydroxytryptophan-induced behavioral symptoms in mice in vivo, and (3) their ability to block the uptake of serotonin, norepinephrine and/or dopamine by synaptosomal rat brain cells in vitro. The ability of the active combinations to counteract reserpine hypothermia in mice in vivo can be determined according to the methods described in U.S. Pat. No. 4,029,731. The activity of the active combinations as antidepressants and related pharmacological properties also can be determined by methods (4)-(8)) below. Specifically, activity can be determined by studying (4) their ability to reverse the stress-induced decrease in sucrose intake in rodents described in Papp, M. et al., European Journal of Pharmacology, 261, 141-147 (1994), (5) learned helplessness paradigm described in Martin P et al., Life Sciences, 48, 2505-2511 (1991), (6) reversing the behavioral deficits of olfactory bulbectomized rats described in Broekkamp C L et al., Pharmacology, Biochemistry and Behavior, 13, 643-646 (1980), (7) increasing down-regulation or desensitization of beta-adrenergic receptors described in Mishra R. et al., Neuropharmacology, 19, 983-987 (1980), and (8) increasing extracellular levels of serotonin, norepinephrine, and/or dopamine in the prefrontal cortex of freely-moving rodents by in vivo dialysis described in Millan M J et al., European Journal of Neuroscience, 12, 1079-1095 (2000).

It should be understood that the present invention is not limited to the embodiments described herein. Numerous modifications can be made by one skilled in the art having the benefits of the teachings given here. Such modifications should be taken as being encompassed within the scope of the present invention as set forth in the appended claims.

What is claimed is:

1. A pharmaceutical composition comprising
(i) a dopamine agonist or a pharmaceutically acceptable salt thereof,
(ii) a compound of the formula I 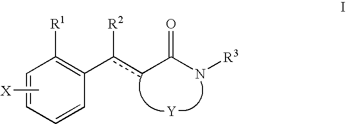
wherein R¹ is a group of the formula G¹, G², G³, G ⁴, G⁵, G or G⁷ depicted below, 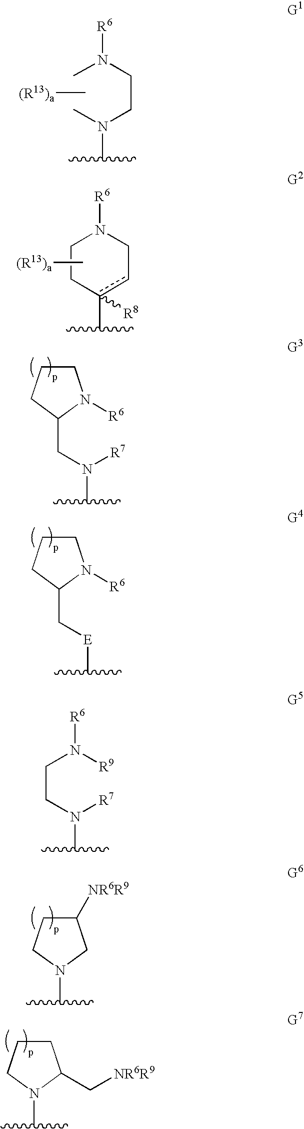
a is zero to eight;
each R¹³ is, independently, (C₁-C₄)alkyl or a (C₁-C₄)methylene bridge from one of the ring carbons of the piperazine or piperidine ring of G¹ or G², respectively, to the same or another ring carbon or a ring nitrogen of the piperazine or piperidine ring of G¹ or G², respectively, having an available bonding site, or to a ring carbon of R⁶ having an available bonding site;
E is oxygen, sulfur, SO or SO₂;
X is hydrogen, chloro, fluoro, bromo, iodo, cyano, (C₁-C₆)alkyl, hydroxy trifluoromethyl, (C₁-C₆)alkoxy, —SOₜ(C₁-C₆)alkyl wherein t is zero one or two, —CO₂R¹⁰ or —CONR¹¹R¹²,
Y is an optionally substituted (C₁-C₄) heteroalkyl bridge that, together with the atoms to which it is attached, forms a five to seven membered heterocycle containing two to four heteroatoms selected from the group consisting of 1,3-oxazolidin-4-on-5-yl, 1,3-oxazolidin-2,4-dion-5-yl, 4,5-dihydro-1,2-oxazolidin-3-on-4-yl, 1,3-thiazolidin-4-on-5-yl, 1,3-thiazolidin-2,4-dion-5-yl, 1,3-pyrazolidin-4-on-5-yl, 1,3-imidazolidin-2,4-d ion-5-yl, 1,2-pyrazolidin-3-on-4-yl, 1,2-thiazolidin-1,1,3-trion-4-yl, 1,2-thiazolidin-3-on-4-yl, tetrahydro-1,2-oxazin-3-on-4-yl, tetrahydro-1,3-oxazin-4-on-5-yl, tetrahydro-1,3-oxazin-2,4-dion-5-yl, morpholin-3-on-2-yl, morpholin-3,5-dion-2-yl, 2,3-dihydro-1,4-oxazin-3-on-2-yl, tetrahydro-1,3-thiazin-4-on-5-yl, tetrahydro-1,3-thiazin-2,4-d ion-5-yl, tetrahydro-1,2-thiazin-3-on-4-yl, thiomorpholin-3-on-2-yl, thiomorpholin-3,5-dion-2-yl, 2,3-dihydro-1,4-thiazin-3-on-2-yl, hexahydro-1,2-diazin-3-on-4-yl, 4,5-dihydro-2H-pyridazin-3-on-4-yl, hexahydro-1,3-diazin-4-on-5-yl, hexahydro-1,3-diazin-2,4-dion-5-yl, piperazin-2-on-3-yl, piperazin-2,6-dion-3-yl, tetrahydro-1,3,4-thiadiazin-5-on-6-yl, 5,6-dihydro-1,3,4-thiadiazin-5-on-6-yl, 1,3,4-oxadiazin-5-on-6-yl, 5,6-dihydro-1,2,4-oxadiazin-5-on-6-yl, tetrahydro-1,2,4-oxadiazin-5-on-6-yl, 1,2,4-triazin-5-on-6-yl, tetrahydro-1,2,4-oxadiazin-5-on-6-yl, 5,6-dihydro-1-2,4-oxadiazin-5-on-6-yl, 1,2,4-oxadiazin-3,5-dion-6-yl, 1,2,4-trazin-6-on-5-yl, hexahydro-1,2-oxazepin-3-on-2-yl, hexahydro-1,3-oxazepin-4-on-5-yl, hexahydro-1,4-oxazepin-3-on-2-yl, hexahydro-1,4-oxazepin-3,5-dion-2-yl, hexahydro-1,4-oxazepin-3,5-dion-6-yl, 2,3,5,6-tetrahydro-1-4-oxazepin-5,7-dion-6-yl, hexahydro-1,4-oxazepin-5-on-6-yl, hexahydro-1,3-oxazepin-2,4-d ion-5-yl, hexahydro-1,2-thiazepin-3-on-4-yl, hexahydro-1,4-thiazepin-3-on-2-yl, 2,3,4,5-tetrahydro-1,4-thiazepin-3-on-2-yl, hexahydro-1,4-thiazepin-3,5-dion-2-yl, hexahydro-1,4-thiazepin-3,5-dion-6-yl, 2,3,6,7-tetrahydro-1,4-thiazepin-5-on-6-yl, 6,7-dihydro-1,4-thiazepin-5-on-6-yl, hexahydro-1,3-thiazepin-2,4-dion-5-yl, hexahydro-1,2-diazepin-3-on-4-yl, hexahydro-1,3-diazepin-2,4-dion-5-yl, hexahydro-1,4-diazepin-2-on-3-yl, hexahydro-1,4-diazepin-5-on-6-yl, hexahydro-1,4-diazepin-5,7-dion-6-yl, hexahydro-1,3,5-thiadiazepin-3-on-7-yl, 4,5,6,7-tetrahydro-1-3,5-thiadiazepin-6-on-7-yl, and 2,3,5,6-tetrahydro-1,2,4-triazepin-3,5-dion-7-yl; wherein the substituents on any of the carbon atoms capable of supporting an additional bond, of said (C₁-C₄) heteroalkyl bridge, are chloro, fluoro, (C₁-C₆)alkyl, (C₁-C₆)alkoxy, trifluoromethyl or cyano; wherein the substituents on any of the nitrogen atoms capable of supporting an additional bond, of said (C₁-C₄) heteroalkyl bridge, are (C₁-C₆)alkyl or trifluoromethyl,
R² is hydrogen, (C₁-C₄)alkyl, phenyl or naphthyl, wherein said phenyl or naphthyl may optionally be substituted with one or more substituents independently selected from the group consisting of chloro, fluoro, bromo, iodo, (C₁-C₆)alkyl, (C₁-C₆)alkoxy, trifluoromethyl, cyano and —SOₖ(C₁-C₆)alkyl wherein k is zero, one or two;
R³ is—(CH₂)ₘB, wherein m is zero, one, two or three and B is hydrogen, phenyl, naphthyl or a 5 or 6 membered heteroaryl group containing from one to four hetero-atoms in the ring, and wherein each of the foregoing phenyl, naphthyl and heteroaryl groups may optionally be substituted with one or more substituents independently selected from the group consisting of chloro, fluoro, bromo, iodo, (C₁-C₆)alkyl, (C₁-C₆)alkoxy, (C₁-C₆) alkoxy-(C₁-C₆)alkyl-, trifluoromethyl, trifluoromethoxy, cyano, hydroxy, —COOH and —SOₙ(C₁-C₆)alkyl wherein n is zero, one or two;
R⁶ is selected from the group consisting of hydrogen, (C₁-C₆)alkyl optionally substituted with (C₁-C₆)alkoxy or one to three fluorine atoms, or [(C₁-C₄)alkyl]aryl wherein the aryl moiety is phenyl, naphthyl, or heteroaryl-(CH₂)_{q}—, wherein the heteroaryl moiety is selected from the group consisting of pyridyl, pyrimidyl, benzoxazolyl, benzothiazolyl, benzisoxazolyl and benzisothiazolyl and q is zero, one, two, three or four, and wherein said aryl and heteroaryl moieties may optionally be substituted with one or more substituents independently selected from the group consisting of chloro, fluoro, bromo, iodo, (C₁-C₆)alkyl, (C₁-C₆)alkoxy, trifluoromethyl, cyano and —SO_{g}(C₁-C₆)alkyl, wherein g is zero, one or two;
R⁷ is selected from the group consisting of hydrogen, (C₁-C₆)alkyl, [(C₁-C₄)alkyl]aryl wherein the aryl moiety is phenyl, naphthyl, or heteroaryl-(CH₂)ᵣ—, wherein the heteroaryl moiety is selected from the group consisting of pyridyl, pyrimidyl, benzoxazolyl, benzothiazolyl, benzisoxazolyl and benzisothiazolyl and r is zero, one, two, three or four, and wherein said aryl and heteroaryl moieties may optionally be substituted with one or more substituents independently selected from the group consisting of chloro, fluoro, bromo, iodo, (C₁-C₆)alkyl, (C₁-C₆)alkoxy, trifluoromethyl, —C(═O)—(C₁-C₆)alkyl, cyano and —SOⱼ(C₁-C₆)alkyl, wherein j is zero, one or two;
or R⁶ and R⁷ taken together form a 2 to 4 carbon chain;
R⁸ is hydrogen or (C₁-C₃)alkyl;
R⁹ is hydrogen or (C₁-C₆)alkyl;
or R⁶ and R⁹, together with the nitrogen atom to which they are attached, form a 5 to 7 membered heteroalkyl ring that may contain from zero to four heteroatoms selected from the group consisting of nitrogen, sulfur and oxygen;
and p is one, two, or three;
each of R¹⁰, R¹¹ and R¹² is selected, independently, from the radicals set forth in the definition of R²; or R¹¹ and R¹², together with the nitrogen to which they are attached, form a 5 to 7 membered heteroalkyl ring that may contain from zero to four heteroatoms selected from the group consisting of nitrogen, sulfur and oxygen, and
the broken lines indicate optional double bonds, with the proviso that when the broken line in G² is a double bond that R⁸ is absent;
or a pharmaceutically acceptable salt thereof, and optionally
(iii) a pharmaceutically acceptable carrier.

2. A composition according to claim 1, wherein R¹ is 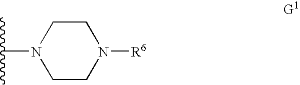
R⁶ is methyl and R² is hydrogen.

3. A composition according to claim 2, wherein Y together with the atoms to which it is attached forms thiomorpholin-3-on-2-yl.

4. A composition according to claim 1 wherein R³ is phenyl or—(CH₂)-phenyl wherein said phenyl groups optionally substituted with one or more substituents independently selected from the group consisting of chloro, fluoro, bromo, iodo, (C₁-C₆)alkyl, (C₁-C₆)alkoxy, (C₁-C₆)alkoxy-(C₁-C₆)alkyl-, trifluoromethyl, trifluoromethoxy, cyano, hydroxy, —COOH and —SOₙ(C₁-C₆)alkyl wherein n is zero, one or two.

5. A composition according to claim 2, wherein R³ is phenyl or—(CH₂)-phenyl wherein said phenyl groups optionally substituted with one or more substituents independently selected from the group consisting of chloro, fluoro, bromo, iodo, (C₁-C₆)alkyl, (C₁-C₆)alkoxy, (C₁-C₆) alkoxy-(C₁-C₆)alkyl-, trifluoromethyl, trifluoromethoxy, cyano, hydroxy, —COOH and —SOₙ(C₁-C₆)alkyl wherein n is zero, one or two.

6. A composition according to claim 3, wherein R³ is phenyl or—(CH₂)-phenyl wherein said phenyl groups optionally substituted with one or more substituents independently selected from the group consisting of chloro, fluoro, bromo, iodo, (C₁-C₆)alkyl, (C₁-C₆)alkoxy, (C₁-C₆)alkoxy-(C₁-C₆)alkyl-, trifluoromethyl, trifluoromethoxy, cyano, hydroxy, —COOH and —SOₙ(C₁-C₆)alkyl wherein n is zero, one or two.

7. A composition according to claim 1, wherein said compound of the formula I is selected from the group consisting of:
4-benzyl-2-[2-(4-methylpiperazin-1-yl)-benzylidene]-thiomorpholin-3-one;
4-(3,4-dichlorobenzyl)-2-[2-(4-methylpiperazin-1-yl)-benzylidene]-thiomorpholin-3-one;
2-[2-(4-methylpiperazin-1-yl)-benzylidene]-4-(4-trifluoromethylphenyl)-thio morpholin-3-one;
2-[2-(4-methylpiperazin-1-yl)-benzylidene]-thiomorpholin-3-one;
4-(3,4-dichlorophenyl)-2-[2-fluoro-6-(4-methylpiperazin-1-yl)-benzyl idene]-thiomorpholin-3-one;
4-(3,4-dichlorophenyl)-2-[2-(4-methylpiperazin-1-yl)-benzylidene]-thiomorpholin-3-one;
4-(3,4-dichlorophenyl)-2-[2-(4-methylpiperazin-1-yl)-benzyl]-thiomorpholin-3-one;
4-methyl-2-[2-(4-methylpiperazin-1-yl)-benzyl idene]-thiomorpholin-3-one;
4-(3,4-dichlorophenyl)-2-(2-piperazin-1-ylbenzylidene)-thiomorpholin-3-one;
or a pharmaceutically acceptable salt thereof.

8. A composition according to claim 1 wherein Y together with the atoms to which it is attached forms an optionally substituted 1-oxo-thiomorpholin-3-on-2-yl.

9. A composition according to claim 1, wherein the compound of formula I is selected from the group consisting of
4-Benzyl-2-[2-(4-methylpiperazin-1-yl)-benzylidene]-1,1-dioxothiomorpholin-3-one;
4-(3,4-Dichlorophenyl)-2-[3-fluoro-2-(4-methylpiperazin-1-yl)-benzylidene]-thiomorpholin-3-one;
4-(3,4-Dichlorophenyl)-2-[5-fluoro-2-(4-methylpiperazin-1-yl)-benzyl idene]-thiomorpholin-3-one;
4-(3,4-Dichlorophenyl)-2-[2-(4-methylpiperazin-1-yl)-5-trifluoromethyl-benzylidene]-thiomorpholin-3-one;
4-(3,4-Dichlorophenyl)-2-{2-[4-(2-methoxyethyl)piperazin-1-yl]-benzyl idene} thiomorpholin-3-one;
4-(3,4-Dichlorophenyl)-2-[2-(4-isopropylpiperazin-1-yl)-benzylidene]-thiomorpholin-3-one;
4-(3,4-Dichlorophenyl)-2-[2-(4-ethylpiperazin-1-yl)-benzylidene]-thiomorpholin-3-one;
4-(4-Chlorophenyl)-2-[2-(4-methylpiperazin-1-yl)-benzylidene]-thiomorpholin-3-one;
4-(3-Chlorophenyl)-2-[2-(4-methylpiperazin-1-yl)-benzylidene]-thiomorpholin-3-one;
2-[2-Chloro-6-(4-methylpiperazin-1-yl)-benzyl idene]-4-(3,4-dichlorophenyl)-thiomorpholin-3-one;
4-(3,4-Dichlorophenyl)-2-[2-(4-methylpiperazin-1-yl)-4-trifluoromethyl-benzyl idene]-thiomorpholin-3-one;
4-(3,4-Dichlorophenyl)-2-[2-(4-methylpiperazin-1-yl)-benzylidene]-1-oxo-thi omorpholin-3-one;
4-(3,4-Dichlorophenyl)-2-(5-fluoro-2-piperazin-1-yl-benzylidene)-thiomorpholin-3-one;
4-(3,4-Dichlorophenyl)-2-[3,6-difluoro-2-(4-methylpiperazin-1-yl)-benzylidene]-thiomorpholin-3-one;
4-(3,4-Dichlorophenyl)-2-[2-(3,5-dimethylpiperazin-1-yl)-benzylidene]-thiomorpholin-3-one;
4-Phenyl-2-[2-(3,4,5-trimethylpiperazin-1-yl)-benzylidene]-thiomorpholin-3-one;
2-[5-Fluoro-2-(4-methylpiperazin-1-yl)-benzylidene]-4-phenyl-thiomorpholin-3-one;
4-Benzo[1,3]dioxol-5-yl-2-[2-(3,5-dimethylpiperazin-1-yl)-benzylidene]-thiomorpholin-3-one;
2-[2-(4-tert-Butylpiperazin-1-yl)-benzylidene]-4-(3,4-dichlorophenyl)-thiomorpholin-3-one;
3-[4-(3,4-Dichlorophenyl)-3-oxo-thiomorpholin-2-ylidenemethyl]-6-dimethylamino-2-(4-methylpiperazin-1-yl)-benzonitrile;
4-(3,4-Dichlorophenyl)-2-[2-(3,4,5-trimethylpiperazin-1-yl)-benzylidene]-thiomorpholin-3-one;
4-(3,4-Dichlorophenyl)-2-[5-methyl-2-(4-methylpiperazin-1-yl)-benzyl idene]-thiomorpholin-3-one;
2-[4-Chloro-2-(4-methylpiperazin-1-yl)-benzylidene]-4-(3,4-dichlorophenyl)-thiomorpholin-3-one;
4-(3,4-Difluorophenyl)-2-[2-(3,5-dimethylpiperazin-1-yl)-benzyl idene]-thiomorpholin-3-one;
4-(2,4-Difluorophenyl)-2-[2-(3,5-dimethylpiperazin-1-yl)-benzyl idene]-thiomorpholin-3-one;
2-[4-Bromo-2-(4-methylpiperazin-1-yl)-benzylidene]-4-(3,4-dichlorophenyl)-thiomorpholin-3-one;
4-(3,4-Dichlorophenyl)-2-[2-(1-methylpyrrolidin-2-ylmethoxy)-benzylidene]-thiomorpholin-3-one;
4-(3,5-Dichlorophenyl)-2-[2-(3,5-dimethylpiperazin-1-yl)-benzylidene]-thiomorpholin-3-one;
4-(3,4-Difluorophenyl)-2-[2-(3,4,5-trimethylpiperazin-1-yl)-benzylidene]-thiomorpholin-3-one;
4-(3,4-Dichlorophenyl)-2-[2-(octahydropyrido[1,2-a]pyrazin-2-yl)-benzylidene]-thiomorpholin-3-one;
2-[2-(4-Cyclopropylpiperazin-1-yl)-benzylidene]-4-pyridin-3-yl-thiomorpholin-3-one;
2-[2-(4-Cyclopropylpiperazin-1-yl)-benzylidene]-4-(3,4-difluorophenyl)-thiomorpholin-3-one;
2-[2-(4-Cyclopropylpiperazin-1-yl)-benzylidene]-4-(3,5-dichlorophenyl)-thiomorpholin-3-one;
4-(3,4-Difluorophenyl)-2-[2-(2,5-dimethylpiperazin-1-yl)-benzylidene]-thiomorpholin-3-one;
4-(3,5-Dichlorophenyl)-2-[2-(2,5-dimethylpiperazin-1-yl)-benzylidene]-thiomorpholin-3-one;
4-(3,4-Dichlorophenyl)-2-[2-(3-methylaminopyrrolidin-1-yl)-benzylidene]-thiomorpholin-3-one;
4-(3,4-Difluorophenyl)-2-[2-(2,4,5-trimethylpiperazin-1-yl)-benzylidene]-thiomorpholin-3-one;
4-Benzo[1,3]dioxol-5-yl-2-[2-(4-cyclopropylpiperazin-1-yl)-benzylidene]-thiomorpholin-3-one;
2-[2-(3,5-Dimethylpiperazin-1-yl)-benzylidene]-4-(4-fluorophenyl)-thiomorpholin-3-one;
4-Benzo[1,3]dioxol-5-yl-2-[2-(2,5-dimethylpiperazin-1-yl)-benzylidene]-thio morpholin-3-one;
2-[2-(3,5-Dimethylpiperazin-1-yl)-benzylidene]-4-phenylthiomorpholin-3-one;
4-(3,4-Dichlorophenyl)-2-[2-(4-methylpiperazin-1-yl)-benzylidene]-thiomorpholin-3-one;
4-(3,4-Dichlorophenyl)-2-[2-(3-dimethylaminopyrrolidin-1-yl)-benzylidene]-thiomorpholin-3-one;
4-(3,4-Dichlorophenyl)-2-[2-(4-methyl-[1,4]diazepan-1-yl)-benzylidene]-thiomorpholin-3-one;
4-(3,4-Dichlorophenyl)-2-[2-(2,4,6-trimethylpiperazin-1-yl)-benzylidene]-thiomorpholin-3-one; and
2-[2-(4-Cyclopropylpiperazin-1-yl)-benzyl idene]-4-(3,4-dichlorophenyl)-thiomorpholin-3-one.

10. A composition according to claim 1, wherein the compound of formula I is selected from the group consisting of 4-benzyl-2-[2-(4-methylpiperazin-1-yl)-benzylidene]-thiomorpholin-3-one, 4-(3,4-dichloro-benzyl)-2-[2-(4-methylpiperazin-1-yl)-benzylidene]-thiomorpholin-3-one, and 2-[2-(4-methylpiperazin-1-yl)-benzylidene]-4-(4-trifluoromethyl-phenyl)-thiomorpholin-3-one.

11. The pharmaceutical composition of claim 1, wherein the dopamine agonist is a compound having the formula 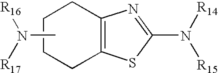
or a pharmaceutically acceptable acid addition salt thereof with an inorganic or organic acid,
wherein R₁₄ represents a hydrogen atom, an alkyl group having 1 to 6 carbon atoms, an alkenyl or alkynyl group each having 3 to 6 carbon atoms, an alkanoyl group having 1 to 6 carbon atoms, a phenyl alkyl or phenyl alkanoyl group having 1 to 3 carbon atoms in the alkyl part, wherein each phenyl may be substituted by 1 or 2 halogen atoms, R₁₅ represents a hydrogen atom or an alkyl group with 1 to 4 carbon atoms, R₁₆ represents a hydrogen atom, an alkyl group with 1 to 7 carbon atoms, a cycloalkyl group having 3 to 7 carbon atoms, an alkenyl or alkynyl group having 3 to 6 carbon atoms, an alkanoyl group having 1 to 7 carbon atoms, a phenyl alkyl or phenyl alkanoyl group having 1 to 3 carbon atoms in the alkyl part, wherein each phenyl may be substituted by fluorine, chlorine or bromine atoms, R₁₇ represents a hydrogen atom, an alkyl group with 1 to 4 carbon atoms, an alkenyl or alkynyl group having 3 to 6 carbon atoms, or R₁₆ and R₁₇ together with the nitrogen atom between them represent a pyrrolidino, piperidino, hexamethyleneimino or morpholino group.

12. The pharmaceutical composition of claim 1, wherein the dopamine agonist is a compound having the formula 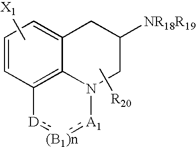
or a pharmaceutically acceptable salt thereof,
wherein R₁₈, R₁₉, and R₂₀ are each independently hydrogen, C₁₋₆ alkyl, C₂₋₆ alkenyl, or C₂₋₆ alkynyl, C₃₋₁₀ cycloalkyl, or R₁₈ and R₁₉ are joined to form together with the nitrogen of NR₁₈R₁₉ a C₃₋₇ cyclic amine which can contain in addition to said nitrogen one or more heteroatoms selected from the group consisting of N, S and O; X₁ is hydrogen, C₁₋₆ alkyl, halogen, hydroxy, C₁₋₆ alkoxy, cyano, carboxamide, carboxyl, or C₁-C₆ alkoxycarbonyl; A₁ is SO₂, N, CH, CH₂, CHCH₃, C═O, C═S, CHSCH₃, C═NH, CNH₂, CNHCH₃, CNHCOOCH₃, or CNHCN; B₁ is CH₂, CH, C═O, N, NH or N—CH₃; n is 0 or 1; and D is CH, CH₂, C═O, O, N, NH or N—CH₃.

13. A pharmaceutical composition according to claim 1, wherein the dopamine agonist is selected from the group consisting of cabergoline, sumanirole and pramipexole.

14. A pharmaceutical composition according to claim 13, wherein the compound of formula I is selected from the group consisting of 4-benzyl-2-[2-(4-methylpiperazin-1-yl)-benzylidene]-thiomorpholin-3-one, 4-(3,4-dichloro-benzyl)-2-[2-(4-methylpiperazin-1-yl)-benzylidene]-thiomorpholin-3-one, and 2-[2-(4-methylpiperazin-1-yl)-benzylidene]-4-(4-trifluoromethyl-phenyl)-thiomorpholin-3-one.

15. A method for treating a disorder or condition selected from the group consisting of hypertension, depression, generalized anxiety disorder, phobias, posttraumatic stress syndrome, avoidant personality disorder, sexual dysfunction, eating disorders, obesity, chemical dependencies, cluster headache, migraine, pain, Alzheimer's disease, obsessive-compulsive disorder, panic disorder, memory disorders, Parkinson's diseases, endocrine disorders, vasospasm, cerebellar ataxia, gastrointestinal tract disorders, negative symptoms of schizophrenia, premenstrual syndrome, Fibromyalgia Syndrome, stress incontinence, Tourette's syndrome, trichotillomania, kleptomania, male impotence, cancer, chronic paroxysmal hemicrania and headache in a mammal, comprising administering to a mammal in need of such treatment (i) a dopamine agonist or a pharmaceutically acceptable salt thereof and (ii) a compound of the formula I as defined in claim 1 or a pharmaceutically acceptable salt thereof.

16. The pharmaceutical composition of claim 1, wherein the compound of the formula I or a pharmaceutically acceptable salt thereof is present in a serotonin receptor antagonizing or agonizing effective amount.

17. The pharmaceutical composition of claim 1, wherein (i) and (ii) are present in amounts such that the combination of (i) and (ii) is effective in treating a disorder or condition selected from the group consisting of hypertension, depression, generalized anxiety disorder, phobias, posttraumatic stress syndrome, avoidant personality disorder, sexual dysfunction, eating disorders, obesity, chemical dependencies, cluster headache, migraine, pain, Alzheimer's disease, obsessive-compulsive disorder, panic disorder, memory disorders, Parkinson's diseases, endocrine disorders, vasospasm, cerebellar ataxia, gastrointestinal tract disorders, negative symptoms of schizophrenia, premenstrual syndrome, Fibromyalgia Syndrome, stress incontinence, Tourette's syndrome, trichotillomania, kleptomania, male impotence, cancer, chronic paroxysmal hemicrania and headache in a mammal.

18. The method of claim 15, wherein the compound of the formula I or a pharmaceutically acceptable salt thereof is present in a serotonin receptor antagonizing or agonizing effective amount.

19. The method of claim 15, wherein (i) and (ii) are present in amounts such that the combination of (i) and (ii) is effective in treating the disorder or condition.

20. A pharmaceutical composition comprising (i) a dopamine agonist or a pharmaceutically acceptable salt thereof, (ii) a compound of the formula I or a pharmaceutically acceptable salt thereof, (iii) optionally a pharmaceutically acceptable carrier, and (iv) a 5-HT_{1A} antagonist, or a pharmaceutically acceptable salt thereof, wherein (i), (ii) and (iv) are present in amounts such that the combination of (i), (ii) and (iv) is effective in treating a disorder or condition selected from the group consisting of hypertension, depression, generalized anxiety disorder, phobias, posttraumatic stress syndrome, avoidant personality disorder, sexual dysfunction, eating disorders, obesity, chemical dependencies, cluster headache, migraine, pain, Alzheimer's disease, obsessive-compulsive disorder, panic disorder, memory disorders, Parkinson's diseases, endocrine disorders, vasospasm, cerebellar ataxia, gastrointestinal tract disorders, negative symptoms of schizophrenia, premenstrual syndrome, Fibromyalgia Syndrome, stress incontinence, Tourette's syndrome, trichotillomania, kleptomania, male impotence, cancer, chronic paroxysmal hemicrania and headache in a mammal.

21. A method for treating a disorder or condition that can be treated by enhancing serotonergic neurotransmission in a mammal, comprising administering to a mammal in need of such treatment (i) a dopamine agonist or a pharmaceutically acceptable salt thereof and (ii) a compound of the formula I as defined in claim 1 or a pharmaceutically acceptable salt thereof.

22. The method of claim 21, further comprising administering to the mammal in need of such treatment a 5-HT_{1A} antagonist, or a pharmaceutically acceptable salt thereof, wherein (i), (ii) and the 5-HT_{1A} antagonist or pharmaceutically acceptable salt thereof are present in amounts such that the combination of (i), (ii), and the 5-HT_{1A} antagonist or pharmaceutically acceptable salt thereof is effective in treating such disorder or condition.

23. A method for enhancing serotonergic neurotransmission in a mammal, comprising administering to a mammal in need of such treatment (i) a dopamine agonist or a pharmaceutically acceptable salt thereof and (ii) a compound of the formula I as defined in claim 1 or a pharmaceutically acceptable salt thereof.
